SEQUENCE LISTING (11 sequences)

SEQ ID NO: 1 - protein, 13 aa, Artificial Sequence
  Synthetic peptide
VESKCGPPCPSCP

SEQ ID NO: 2 - protein, 13 aa, Artificial Sequence
  Synthetic peptide
VESKYCPPCPSCP

SEQ ID NO: 3 - protein, 13 aa, Artificial Sequence
  Synthetic peptide
VESKCCPPCPSCP

SEQ ID NO: 4 - protein, 13 aa, Artificial Sequence
  Synthetic peptide
VESKCCPPCPPCP

SEQ ID NO: 5 - protein, 16 aa, Artificial Sequence
  Synthetic peptide
VEPKSCDKTHTCPPCP

SEQ ID NO: 6 - protein, 13 aa, Artificial Sequence
  Synthetic peptide
VERKCCVECPPCP

SEQ ID NO: 7 - protein, 13 aa, Artificial Sequence
  Synthetic peptide
VESKYGPPCPSCP

SEQ ID NO: 8 - protein, 13 aa, Artificial Sequence
  Synthetic peptide
VESKYGPPCPPCP

SEQ ID NO: 9 - protein, 13 aa, Artificial Sequence
  Synthetic peptide
VESKCGPPCPPCP

SEQ ID NO: 10 - protein, 13 aa, Artificial Sequence
  Synthetic peptide
VESKYCPPCPPCP

SEQ ID NO: 11 - protein, 327 aa, Artificial Sequence
  Synthetic peptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0270325 A1
(43) Pub. Date: Aug. 28, 2025

(54) STABILIZED IGG4 ANTIBODIES AND USES THEREOF

(71) Applicant: MEDIMMUNE, LLC, Gaithersburg, MD (US)

(72) Inventors: Michael W. HANDLOGTEN, Gaithersburg, MD (US); William DALL'ACQUA, Gaithersburg, MD (US); Sanjeev AHUJA, Gaithersburg, MD (US); Li PENG, Gaithersburg, MD (US)

(21) Appl. No.: 17/907,683

(22) PCT Filed: Mar. 29, 2021
(86) PCT No.: PCT/IB2021/052589

§ 371 Date: Sep. 29, 2022

Related U.S. Application Data
(60) Provisional application No. 63/002,631, filed on Mar. 31, 2020.

(51) Int. Cl. C07K 16/28

(52) U.S. Cl. CPC C07K 16/283; C07K 2317/24; C07K 2317/31; C07K 2317/53; C07K 2317/92; C07K 2317/94

(57) ABSTRACT

The disclosure relates to an antibody of the class IgG4 comprising two heavy chains, wherein each of the heavy chains comprises a human IgG4 constant region comprising a mutation. The disclosure further relates to methods of treating a disorder or condition using the IgG4 antibody comprising two heavy chains, wherein each of the heavy chains comprises a human IgG4 constant region comprising a mutation.

### FIELD OF THE DISCLOSURE

The disclosure relates to an antibody comprising two heavy chains, wherein each of the heavy chains comprises a human IgG4 constant region comprising a mutation. The disclosure further relates to methods of treating a disorder or condition using the antibody comprising two heavy chains, wherein each of the heavy chains comprises a human IgG4 constant region comprising a mutation.

### BACKGROUND

IgG4s are dynamic molecules that undergo a process called Fab-arm exchange. Disulfide bonds between heavy chains are transiently reduced, resulting in two half antibodies that reform intact antibodies with other IgG4 half antibodies. In vivo, therapeutic IgG4s can recombine with endogenous IgG4s resulting in a heterogeneous mixture of bispecific antibodies. A related issue that can occur for any therapeutic protein during manufacturing is interchain disulfide bond reduction. For IgG4s, this primarily results in high levels of half-mAb that persist through purification processes. The S228P mutation has been used to prevent half-mAb formation for IgG4s. However, IgG4s with the S228P mutation are subject to half-mAb formation in reducing environments. Thus, there remains a need for antibodies that do not undergo reduction during manufacturing and/or form half-antibodies in vivo.

### SUMMARY

Provided herein is an IgG4 antibody comprising two heavy chains, wherein each of the heavy chains comprises a human IgG4 constant region comprising at least one mutation at residue 219 or 220 of the heavy chain according to EU index of numbering.

Also provided herein is an IgG4 antibody comprising two heavy chains, wherein each of the heavy chain comprises a human IgG4 constant region comprising a mutation at residues 219, 220, and 228 of the heavy chain according to EU index of numbering.

Also provided herein is method of treating a subject having a disease or condition comprising administering to the subject a therapeutically effective amount of the IgG4 antibodies of the disclosure.

Also provided herein is a pharmaceutical composition comprising a therapeutically effective amount of the IgG4 antibodies, antibody-drug conjugates, antibody-peptide conjugates, or antibody-protein conjugates of the disclosure, and a pharmaceutically acceptable carrier.

### BRIEF DESCRIPTION OF THE DRAWINGS

FIG. 1 shows the relative stability of IgGs towards reduction by the thioredoxin system. IgG1, IgG2, and IgG4 mAbs were incubated with the components of the thioredoxin system. Samples were taken at the indicated timepoints and intact mAb was quantified using capillary electrophoresis.

FIGS. 2A-2F show intermediate species formed during reduction by the thioredoxin system. IgG1, IgG2, and IgG4 mAbs were incubated with the components of the thioredoxin system. Samples were taken at the indicated time points and mAb fragments were quantified using capillary electrophoresis. For all Figures, intact mAb (LHHL) is on the left axis and fragment species (HHL, HH, and HL) are on the right axis. Free heavy and light chain were not shown to aid in visualization.

FIG. 3 depicts the major intermediates formed for different IgG isotypes when exposed to reducing conditions.

FIG. 4 shows that hinge mutations increased the stability of IgG4 mAbs towards reduction. IgG4 mAbs with the indicated hinge mutations were incubated with the components of the thioredoxin system. Samples were taken at the indicated time points and intact mAb was quantified using capillary electrophoresis. Data represents the mean±SD of triplicate experiments.

FIGS. 5A-5H show that hinge mutations decreased half-antibody formation. IgG4 mAbs with the indicated hinge mutations were incubated with the components of the thioredoxin system. Samples were taken at the indicated time points and mAb fragments were quantified using capillary electrophoresis. Free heavy and light chain were not shown to aid in visualization. Data represents the mean±SD of triplicate experiments.

FIGS. 6A-6C show that hinge mutations did not affect antigen, FcRn, or FcγRIIIa binding in vitro. FIG. 6A shows antigen binding of the IgG4 mAbs with the indicated hinge mutations as compared to the unmodified IgG4 in an ELISA assay. An isotype control demonstrated no binding up to 5000 nM (data not shown). Data represents the mean±SD of duplicate experiments. FIG. 6B shows FcRn and FIG. 6C shows FcγRIIIa binding for IgG4s with hinge mutations as compared to the unmodified IgG4 in AlphaLISA assays. Rituximab was run as a known positive control in the FcγRIIIa AlphaLISA assay. Data represents the mean SD of duplicate independent experiments.

FIGS. 7A-7H show deconvoluted mass spectra for unmodified IgG4 (FIG. 7A), IgG4-S228P (FIG. 7B), IgG4-G220C (FIG. 7C), IgG4-Y219C (FIG. 7D), IgG4-Y219C+G220C (FIG. 7E), IgG4-Y219C+S228P (FIG. 7F), IgG4-G220C+S228P (FIG. 7G), and IgG4-Y219C+G220C+S228P (FIG. 7H).

FIG. 8 shows sequence alignment between the constant heavy chains of IgG1, IgG2 and IgG4.

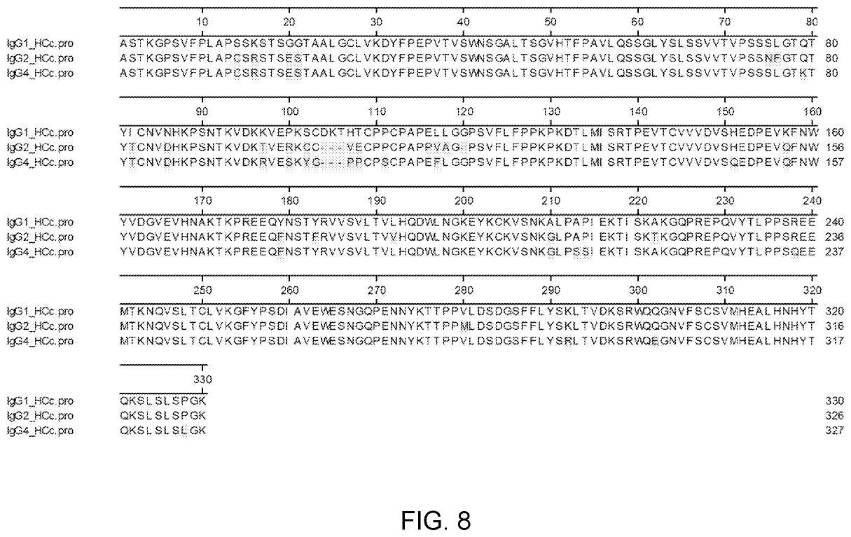

FIGS. 9A-9C show the quantification of Fab-arm exchange under different reducing conditions. FIG. 9A shows hybrid mAb formation for IgG4 mAbs with the indicated hinge mutation after incubation with a second, unmodified IgG4. Hybrid mAb formation was quantified as a percent of total protein using capillary electrophoresis. Data represents the mean±SD of triplicate experiments. FIG. 9B shows an example electropherogram from capillary electrophoresis for unmodified IgG4. FIG. 9C shows the mass spectrometry data for the unmodified IgG4.

### DETAILED DESCRIPTION

The disclosure relates to an antibody comprising two heavy chains, wherein each of the heavy chains comprises a human IgG4 constant region comprising a mutation. The disclosure further relates to methods of treating a disorder or condition using the IgG4 antibody comprising two heavy chains, wherein each of the heavy chains comprises a human IgG4 constant region comprising a mutation.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

The term “antibody” as used herein refers to a protein that is capable of recognizing and specifically binding to an antigen. Ordinary or conventional mammalian antibodies comprise a tetramer, which is typically composed of two identical pairs of polypeptide chains, each pair consisting of one “light” chain (typically having a molecular weight of about 25 kDa) and one “heavy” chain (typically having a molecular weight of about 50-70 kDa). The terms “heavy chain” and “light chain,” as used herein, refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxyl-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}) and a hinge region between C_{H1} and C_{H2}, wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Within full-length light and heavy chains, the variable and constant domains typically are joined by a “J” region of about 12 or more amino acids, with the heavy chain also including a “D” region of about 10 more amino acids. The variable regions of each light/heavy chain pair typically form an antigen-binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

Reference to the numbering of amino acid residues described herein is performed according to the EU numbering system (also described in Kabat et al., Sequences of Proteins of Immunological Interest, 5th Ed. Public Health Service, National Institutes of Health, Bethesda, MD (1991)).

In particular embodiments provided herein is an IgG4 antibody comprising two heavy chains, wherein each of the heavy chains comprises a human IgG4 constant region comprising at least one mutation. In some embodiments, the heavy chain comprises a human IgG4 constant region comprising the amino acid sequence of SEQ ID NO: 11. In other embodiments, the heavy chain comprises a human IgG4 constant region comprising the amino acid sequence of SEQ ID NO: 11 with one or more mutation. In other embodiments, the IgG4 heavy chain constant region comprises the amino acid sequence of SEQ ID NO: 11 with one, two, or three mutations.

In particular embodiments provided herein is an IgG4 antibody comprising two heavy chains, wherein each of the heavy chains comprises a human IgG4 constant region comprising at least one mutation at residue 219 or 220 of the heavy chain according to EU index of numbering. In some embodiments, the human IgG4 constant region comprises a cysteine at residue 219 of the heavy chain. In some embodiments, the human IgG4 constant region comprises a cysteine at residue 220 of the heavy chain. In some embodiments, the human IgG4 constant region comprises a cysteine at residue 219 and a cysteine at residue 220 of the heavy chain. In some embodiments, the human IgG4 constant region further comprises a proline at residue 228 of the heavy chain. In some embodiments, each of the heavy chains comprises a human IgG4 constant region comprising a mutation at residues 219, 220, and 228 of the heavy chain according to EU index of numbering. In some embodiments, the antibody comprises a cysteine at residue 219, a cysteine at residue 220, and a proline at residue 228 of the heavy chain.

In particular embodiments, provided herein is an IgG4 antibody comprising two heavy chains, wherein each of the heavy chains comprises a human IgG4 constant region comprising at least one mutation within the hinge region. The term “hinge” or “hinge region” refers to the flexible polypeptide comprising the amino acids between the first and second constant domains of an IgG4 antibody. A mutation in the hinge region can be generated by methods well known in the art, such as, introducing a modification into a wild type hinge using amino acid insertions, deletions, substitutions, and rearrangements. The mutated hinge regions disclosed herein may be incorporated into a molecule including, but not limited to, antibodies and fragments thereof. In particular embodiments, the IgG4 antibody comprises two heavy chains, wherein each of the heavy chains comprises at least one mutation within the hinge region. In particular embodiments, the IgG4 antibody further comprises two light chains. The amino acid sequence of several mutated hinges are detailed in Table 2. In particular embodiments, the IgG4 antibody comprises two heavy chains, wherein each of the heavy chains comprises a human IgG4 constant region comprising a hinge region comprising an amino acid sequence of VESKCGPPCPSCP (SEQ ID NO: 1), VESKYCPPCPSCP (SEQ ID NO: 2),

(SEQ ID NO: 8)
VESKYGPPCPPCP,
(SEQ ID NO: 9)
VESKCGPPCPPCP,
(SEQ ID NO: 10)
VESKYCPPCPPCP,
(SEQ ID NO: 3)
VESKCCPPCPSCP,
or
(SEQ ID NO: 4)
VESKCCPPCPPCP.

The term “vector,” refers to any molecule (e.g., nucleic acid, plasmid, or virus) that is used to transfer coding information to a host cell. One type of vector is a “plasmid,” which refers to a circular double-stranded DNA molecule into which additional DNA segments may be inserted. Another type of vector is a viral vector, wherein additional DNA segments may be inserted into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell and thereby are replicated along with the host genome. In addition, certain vectors are capable of directing the expression of genes to which they are operatively linked. Such vectors are referred to herein as “expression vectors.”

The antibodies disclosed herein can be conjugated to a drug or therapeutic agent that modifies a given biological response. Suitable drugs include chemical therapeutic agents, a protein or polypeptide possessing a desired biological activity for example, a toxin, a thrombotic agent or an anti-angiogenic agent, or a growth factor. Additionally, the antibody can be conjugated to therapeutic moieties such as a radioactive materials or macrocyclic chelators. In some embodiments, provided herein is an antibody-drug conjugate comprising the IgG4 antibodies disclosed herein.

The IgG4 antibodies of the disclosure can be fused or chemically conjugated to a protein or peptide to generate fusion proteins. The fusion does not necessarily need to be direct, but may occur through linker sequences. In some embodiments provided herein is an antibody-peptide conjugate comprising the IgG4 antibodies disclosed herein. In some embodiments, provided herein is an antibody-protein conjugate comprising the IgG4 antibodies disclosed herein.

The antibodies disclosed herein include bispecific antibodies, human antibodies, humanized antibodies, or chimeric antibodies. In particular embodiments, the bispecific antibody is capable of specifically binding to a first antigen and a second antigen.

The term “specifically binds” refers to the capability of an antibody to bind to a particular molecule or fragment thereof (e.g., antigen). For example, an antibody that specifically binds a molecule or fragment thereof may bind to other molecules with lower affinity as determined by, e.g., immunoassays, BIAcore, or other assays known in the art. In particular, antibodies (or antigen-binding fragments thereof) that specifically bind to at least one molecule or fragment thereof can compete off other molecules that bind non-specifically to the molecule or fragment thereof. The present disclosure encompasses antibodies with multiple specificities (e.g., an antibody with specificity for two or more discrete antigens). For example, a bispecific antibody can bind to two adjacent epitopes on a single target antigen, or can bind to two different antigens.

In some embodiments, the antibody specifically binds erythropoietin, β-amyloid, thrombopoietin, interferon-α (2a and 2b), interferon-β (Ib), interferon-γ, TNFR I (CD120a), TNFR II (CD120b), IL-IR type 1 (CD121a), IL-IR type 2 (CD121b), IL-2, IL2R (CD25), IL-2R-β (CD123), IL-3, IL-4, IL-3R (CD123), IL-4R (CD124), IL-5R (CD125), IL-6R-α (CD126), IL-6R-β (CD130), IL-8, IL-10, IL-I1, IL-15, IL-15BP, IL-15R, IL-20, IL-21, TCR variable chain, RANK, RANK-L, CTLA4, CXCR4R, CCR5R, TGF-β1, -β2, -β3, G-CSF, GM-CSF, MIF-R (CD74), M-CSF-R (CD115), GM-CSFR (CD116), soluble FcRI, sFcRII, sFcRIII, FcRn, Factor VII, Factor VIII, Factor IX, VEGF, α-4 integrin, Cd11a, CD18, CD20, CD38, CD25, CD74, FcαRI, FcεRI, acetyl choline receptor, fas, fasL, TRAIL, hepatitis virus, hepatitis C virus, envelope E2 of hepatitis C virus, tissue factor, a complex of tissue factor and Factor VII, EGFr, HER2, CD4, CD28, VLA-I, 2, 3, or 4, LFA-I, MAC-I, 1-selectin, PSGL-I, ICAM-I, P-selectin, periostin, CD33 (Siglec 3), Siglec 8, TNF, CCL1, CCL2, CCL3, CCL4, CCL5, CCLU, CCL13, CCL17, CCL18, CCL20, CCL22, CCL26, CCL27, CX3CL1, LIGHT, EGF, VEGF, TGFα, HGF, PDGF, NGF, complement, MBL, factor B, a Matrix Metallo Protease, CD32b, CD200, CD200R, OX-40, Killer Immunoglobulin-Like Receptors (KIRs), NKG2D, leukocyte-associated immunoglobulin-like receptors (LAIRs), Iv49, PD-L2, CD26, BST-2, ML-IAP (melanoma inhibitor of apoptosis protein), cathepsin D, CD40, CD40R, CD86, a B cell receptor, CD79, PD-1, or a T cell receptor.

The IgG4 antibodies disclosed herein show advantageous properties. In some embodiments, the antibody has improved stability towards reduction as compared to an IgG4 antibody that does not comprise the mutations. In some embodiments, the antibody has greater than 3-fold stability towards reduction as compared to an IgG4 antibody that does not comprise the mutations. In some embodiments, the antibody has greater than 6-fold stability towards reduction as compared to an IgG4 antibody that does not comprise the mutations. In some embodiments, the antibody has reduced half-antibody formation as compared to an IgG4 antibody that does not comprise the mutations. In some embodiments, the antibody has at least 40% less half-antibody formation as compared to an IgG4 antibody that does not comprise the mutations. In some embodiments, the antibody has at least 80% less half-antibody formation as compared to an IgG4 antibody that does not comprise the mutations.

In particular embodiments, provided herein is a method of treating a subject having a disease or condition comprising administering to the subject a therapeutically effective amount of an IgG4 antibody disclosed herein.

A “disease” or “condition” refers to any condition that would benefit from treatment using the methods of the disclosure. “Disease” and “condition” are used interchangeably herein and include chronic and acute disorders or diseases, including those pathological conditions that predispose a patient to the disorder in question.

The term “subject” is intended to include human and non-human animals, particularly mammals. In certain embodiments, the subject is a human patient.

The terms “treatment” or “treat” as used herein refer to both therapeutic treatment and prophylactic or preventative measures. Those in need of treatment include subjects having a disease or condition as well as those prone to having disease or condition or those for which a disease or condition is to be prevented.

In some embodiments, the methods disclosed herein relate to treating a subject for cancer. In some embodiments, the cancer is melanoma, breast cancer, pancreatic cancer, lung cancer, hepatocellular carcinoma, cholangiocarcinoma or biliary tract cancer, gastric cancer, oesophagus cancer, head and neck cancer, renal cancer, cervical cancer, colorectal cancer, or urothelial carcinoma.

In some embodiments, the methods disclosed herein relate to treating a subject for a neurological disease. In some embodiments, the neurological disease is ischemic stroke, cerebral infarction, neurotrauma, Parkinson's disease, amyotrophic lateral sclerosis (Lou Gehrig's disease), or epilepsy.

In some embodiments, the methods disclosed herein relate to treating a subject for a neurodegenerative disease. In some embodiments, the neurodegenerative disease is Alzheimer's disease, Parkinson's disease, ischemic dementia, or Huntington's disease.

In some embodiments, the methods disclosed herein relate to treating a subject for an autoimmune disease. In some embodiments, the autoimmune disease is multiple sclerosis, pulmonary fibrosis, rheumatoid arthritis, Type 1 diabetes mellitus, Addison's disease, Myasthenia gravis, systemic lupus erythematosus, psoriasis, Grave's disease, Celiac disease, Hashimoto's thyroiditis, vasculitis, or Crohn's disease.

In some embodiments, the methods disclosed herein relate to treating a subject for a cardiovascular disease. In some embodiments, the cardiovascular disease is an arrhythmia, a coronary heart disease, a cerebrovascular disease, a peripheral arterial disease, a rheumatic heart disease, a congenital heart disease, deep vein thrombosis or a pulmonary embolism.

In some embodiments, the methods disclosed herein relate to treating a subject for a metabolic disease. In some embodiments, the metabolic disease is hyperglycemias, insulin resistance, diabetes, dyslipemias, or obesity.

In some embodiments, the methods disclosed herein relate to treating a subject for a respiratory disease. In some embodiments, the respiratory disease is asthma, chronic obstructive pulmonary disease, bronchitis, emphysema, lung cancer, cystic fibrosis, pneumonia or pleural effusion.

In some embodiments, the methods disclosed herein relate to treating a subject for an infection. In some embodiments, the infection is a bacterial or viral infection.

The terms “administration” or “administering” as used herein refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

The terms “co-administered” or “in combination” as used herein refer to simultaneous or sequential administration of multiple compounds or agents. A first compound or agent may be administered before, concurrently with, or after administration of a second compound or agent. The first compound or agent and the second compound or agent may be simultaneously or sequentially administered on the same day, or may be sequentially administered within 1 day, 2 days, 3 days, 4 days, 5 days, 6 days, 1 week, 2 weeks, 3 weeks, or 1 month of each other. In some embodiments, compounds or agents are co-administered during the period in which each of the compounds or agents are exerting at least some physiological effect and/or has remaining efficacy.

The terms “pharmaceutical composition” or “therapeutic composition” as used herein refer to a compound or composition capable of inducing a desired therapeutic effect when properly administered to a subject. In some embodiments, the disclosure provides a pharmaceutical composition comprising a pharmaceutically acceptable carrier and a therapeutically effective amount of at least one antibody, antibody-drug conjugate, antibody-peptide conjugate, or antibody-protein conjugate of the disclosure.

A “therapeutically effective dose” or “therapeutic dose” is an amount sufficient to effect desired clinical results (i.e., achieve therapeutic efficacy). A therapeutically effective dose can be administered in one or more administrations.

The terms “pharmaceutically acceptable carrier” or “physiologically acceptable carrier” as used herein refer to one or more formulation materials suitable for accomplishing or enhancing the delivery of one or more antibodies of the disclosure.

In some embodiments, the IgG4 antibodies disclosed herein may be formulated with a pharmaceutically acceptable carrier, excipient, or stabilizer, as pharmaceutical compositions. In certain embodiments, such pharmaceutical compositions are suitable for administration to a human or non-human animal via any one or more routes of administration using methods known in the art. The term “pharmaceutically acceptable carrier” means one or more non-toxic materials that do not interfere with the effectiveness of the biological activity of the active ingredients. Such preparations may routinely contain salts, buffering agents, preservatives, compatible carriers, and optionally other therapeutic agents. Such pharmaceutically acceptable preparations may also contain compatible solid or liquid fillers, diluents or encapsulating substances which are suitable for administration into a human. Other contemplated carriers, excipients, and/or additives, which may be utilized in the formulations described herein include, for example, flavoring agents, antimicrobial agents, sweeteners, antioxidants, antistatic agents, lipids, protein excipients such as serum albumin, gelatin, casein, salt-forming counterions such as sodium, and the like. These and additional known pharmaceutical carriers, excipients, and/or additives suitable for use in the formulations described herein are known in the art, e.g., as listed in “Remington: The Science & Practice of Pharmacy,” 21st ed., Lippincott Williams & Wilkins, (2005), and in the “Physician's Desk Reference,” 60th ed., Medical Economics, Montvale, N.J. (2005). Pharmaceutically acceptable carriers can be selected that are suitable for the mode of administration, solubility, and/or stability desired or required.

In one embodiment, the formulations of the disclosure are pyrogen-free formulations that are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension, and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one-hour period for intravenous drug applications (The United States Pharmacopeial Convention, Pharmacopeial Forum 26(1): 223 (2000)). In certain embodiments, the endotoxin and pyrogen levels in the composition are less than 10 EU/mg, or less than 5 EU/mg, or less than 1 EU/mg, or less than 0.1 EU/mg, or less than 0.01 EU/mg, or less than 0.001 EU/mg.

When used for in vivo administration, the formulations of the disclosure should be sterile. The formulations of the disclosure may be sterilized by various sterilization methods, including, for example, sterile filtration or radiation. In one embodiment, the formulation is filter sterilized with a presterilized 0.22-micron filter. Sterile compositions for injection can be formulated according to conventional pharmaceutical practice as described in “Remington: The Science & Practice of Pharmacy,” 21st ed., Lippincott Williams & Wilkins, (2005).

In some embodiments, therapeutic compositions can be formulated for particular routes of administration, such as oral, nasal, pulmonary, topical (including buccal and sublingual), rectal, vaginal, and/or parenteral administration. The terms “parenteral administration” and “administered parenterally” as used herein refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection, and infusion. Formulations of the disclosure that are suitable for topical or transdermal administration include powders, sprays, ointments, pastes, creams, lotions, gels, solutions, patches, and inhalants. The antibodies and other actives may be mixed under sterile conditions with a pharmaceutically acceptable carrier, and with any preservatives, buffers, or propellants which may be required (see, e.g., U.S. Pat. Nos. 7,378,110; 7,258,873; and 7,135,180; U.S. Patent Application Publication Nos. 2004/0042972 and 2004/0042971).

The formulations can be presented in unit dosage form and can be prepared by any method known in the art of pharmacy. Actual dosage levels of the active ingredients in the pharmaceutical compositions of the present disclosure may be varied so as to obtain an amount of the active ingredient which is effective to achieve the desired therapeutic response for a particular patient, composition, and mode of administration, without being toxic to the patient (e.g., “a therapeutically effective amount”). The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. These dosages may be administered daily, weekly, biweekly, monthly, or less frequently, for example, biannually, depending on dosage, method of administration, disorder or symptom(s) to be treated, and individual subject characteristics. Dosages can also be administered via continuous infusion (such as through a pump). The administered dose may also depend on the route of administration. For example, subcutaneous administration may require a higher dosage than intravenous administration. As noted above, any commonly used dosing regimen (e.g., 1-10 mg/kg administered by injection or infusion daily or twice a week) may be adapted and suitable in the methods relating to treating human cancer patients.

In particular embodiments, provided herein is a method of in vitro diagnosis of a disease or a condition in a subject. The antibodies disclosed herein can be used in vitro in immunoassays in which they can be utilized in liquid phase or bound to a solid phase carrier. In addition, the antibodies in these immunoassays can be detectably labeled in various ways. Examples of types of immunoassays which can utilize the antibodies disclosed herein are flow cytometry, e.g., FACS, MACS, immunohistochemistry, competitive and non-competitive immunoassays in either a direct or indirect format.

Without limiting the disclosure, a number of embodiments of the disclosure are described herein for the purpose of illustration.

### EXAMPLES

The Examples that follow are illustrative of specific embodiments of the disclosure, and various uses thereof. They are set forth for explanatory purposes only and should not be construed as limiting the scope of the disclosure in any way.

### Materials and Methods

### Stability Towards Reduction

The relative stability of mAbs towards reduction by the thioredoxin system were assessed by spiking purified antibody at 1.2 mg/mL into solutions containing 2 μM thioredoxin (Abcam; Cambridge, MA), 0.1 μM thioredoxin reductase (Cayman Chemical; Ann Arbor, MI), and 0.24 mM NADPH (Millipore-Sigma; St. Louis, MO) in phosphate buffer, pH 7.4, containing 10 mM EDTA. The amount of intact antibody and intermediates formed were quantified using a LabChip GXII Touch HT (Perkin Elmer). Samples were taken at the indicated time points, stored frozen at −80° C., and then analyzed using non-reducing conditions following the standard protocol from Perkin Elmer.

### mAb Expression and Purification

Hinge mutations were all incorporated into the same IgG4 sequence using site directed mutagenesis as previously described (Peng et al., PloS One 7: e36412 (2012); Bezabeh et al., mAbs 9: 240-56 (2017)). Mutated IgG4 sequences were expressed via transient transfection in CHO cells and purified using protein A chromatography followed by size exclusion purification to reduce product aggregates to <2%.

### Antigen Binding ELISA

96-well plates were coated with 4 μg/mL of the target antigen overnight at 4° C. The plates were blocked with a 5% milk solution in PBS with 0.5% Tween 20. The primary mAb was detected with a secondary anti-human IgG4 HRP-conjugated antibody (1:1000 dilution, Invitrogen MH1742). Following addition of tetramethylbenzidine (TMB), the reaction was stopped with 0.2N sulfuric acid and absorbance was read at 450 nm. Between all steps, the wells were washed 4 times with PBS containing 0.05% Tween 20.

### FcRn Binding AlphaLISA

An AlphaLISA® FcRn competition binding assay kit (PerkinElmer, Catalog #AL3095) was used to measure relative FcRn binding. Increasing concentrations of antibody bound to FcRn competitively block the ability of the acceptor and donor beads to proximally interact, which in turn decreases the assay signal. IgG4 antibody samples were prepared to a starting concentration of 100 μg/mL in MES buffer. A ten-point dilution series was prepared and incubated with biotinylated FcRn in a 96-well plate for 45 minutes at room temperature. Streptavidin-coated donor beads and human IgG conjugated acceptor beads were prepared to 20 μg/mL, added to the antibody-FcRn solution, and incubated for 60 minutes at room temperature in the dark. Chemiluminescence was quantified using an Envision spectrometer, and the resulting data was fit with SoftMax software using a 4-parameter fit.

### FcγRIIIa Binding AlphaLISA

An AlphaLISA® FcγRIIIa competition binding assay kit (PerkinElmer, Catalog #AL348) was used to measure relative FcγRIIIa-158V binding. Biotinylated human FcγRIIIa-158V is captured by streptavidin-coated donor beads. In the absence of antibody samples, human IgG Fc region conjugated to acceptor beads binds the FcγRIIIa-158V and brings the donor and acceptor beads into proximity, generating a chemiluminescent emission. IgG4 antibody samples were prepared to a starting concentration of 20 μg/mL in HiBlock buffer and pre-incubated for 30 minutes at room temperature with biotinylated human FcγRIIIa-158V to facilitate binding. Acceptor and donor beads were prepared to 20 μg/mL, then added to the antibody-receptor solution and incubated for 60 minutes at room temperature in the dark. Chemiluminescence was quantified using an Envision spectrometer, and the resulting data was fit with SoftMax software using a 4-parameter fit. Rituximab was run as a known positive control.

### Mass Spectrometry

Intact mass analysis was performed with a Waters SYNAPT G2-Si High Definition Mass Spectrometer in conjunction with a UPLC system (Waters). For non-reducing intact mass analysis, 1 μg of each sample was directly injected onto a reversed phase column (Acquity UPLC BEH300, C4, 1.7 μm, 2.1 mm×50 mm; Waters) and eluted on a linear gradient using a mobile phase A comprising of 0.1% FA and 0.01% TFA in water and a mobile phase B comprising of 0.1% FA and 0.01% TFA in acetonitrile. Mass spectra were collected at a range of 500-4,500 m/z. Molecular mass was determined by deconvolution of the mass data, using the MaxEnt I in MassLynx software (Waters).

### Example 1: Comparison of Reduction Kinetics and Pathways Across IgG Isotypes

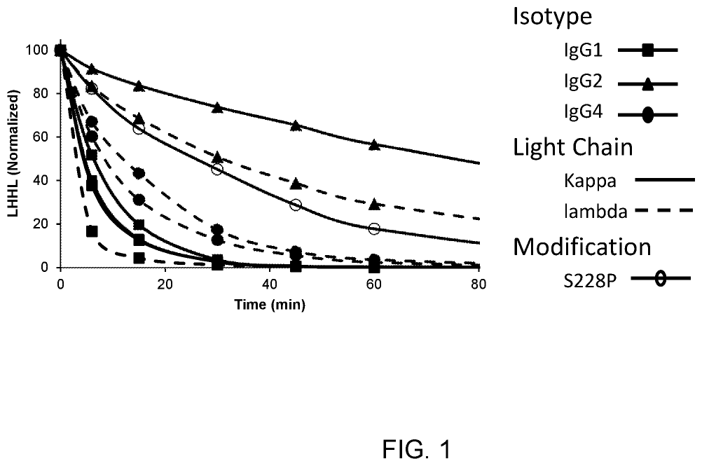

The relative stability towards reduction of a variety of mAbs was tested by incubating each with thioredoxin, thioredoxin reductase, and NADPH. The library of mAbs that were evaluated included IgG1s, IgG2s, and IgG4s. Additionally, four of the mAbs had lambda light chains, five had kappa light chains, and one of the IgG4s had the commonly used S228P mutation. It was found that IgG2 were more stable towards reduction than IgG1s, and that mAbs with kappa light chains were more stable than mAbs with lambda light chains (FIG. 1) (Hutterer et al., mAbs 5: 608-13 (2013)). Interestingly, despite the propensity of IgG4s to form half-mAbs, it was found that IgG4 mAbs were more stable towards reduction than IgG1s. The S228P mutation has been incorporated into several therapeutic IgG4s to prevent the formation of half-mAbs (Kaplon et al., mAbs 11: 219-38 (2019)). As expected, the IgG4 with the S228P mutation was more stable towards reduction than IgG4s with the wild type hinge sequence.

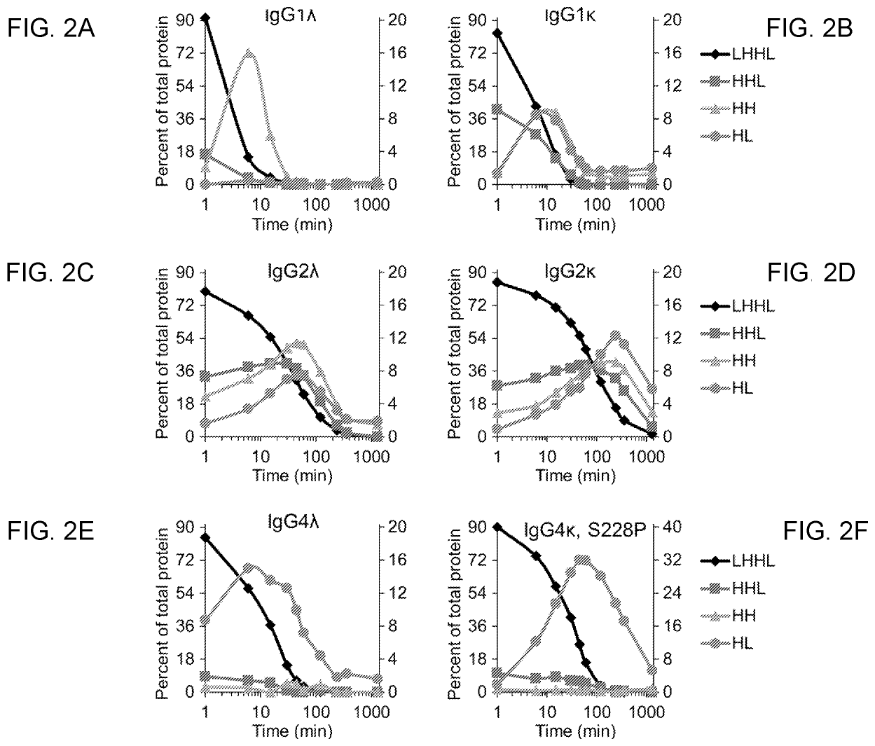

Next, the intermediate species formed for each of the mAbs were examined as they were reduced from intact mAbs to free heavy and light chains by the thioredoxin system. A comparison of a representative set, containing IgG1s, IgG2s, and IgG4s, each with kappa or lambda light chains, is shown in FIG. 2. The results demonstrate that similar intermediate species are formed for IgG1s and IgG2s and that these species are dependent on the light chain type. The dominate intermediate species formed for IgG1 and IgG2 mAbs with lambda light chains are heavy chains dimers (HH) while IgG1 and IgG2 mAbs with kappa light chains form similar or greater amounts of half-mAb (HL) as compared to heavy chain dimer (HH). These results demonstrate that the disulfide bond between a lambda light chain and the heavy chain is more easily reduced than the disulfide bond between a kappa light chain and the heavy chain.

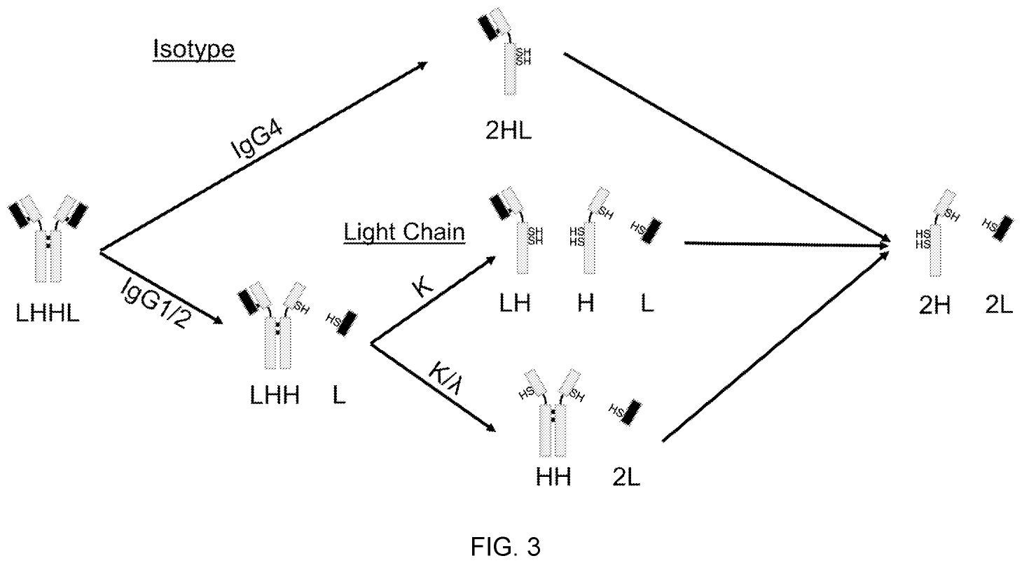

The intermediates formed for the IgG4 mAbs did not depend on the light chain isotype or even the presence of the S228P hinge mutation. As shown in FIG. 2, both IgG4s formed almost exclusively half-mAb (HL) as they were reduced by the thioredoxin system. The IgG4 with the S228P mutation was more stable towards reduction by the thioredoxin system but the results demonstrate that upon reduction, the mAb still formed exclusively half-mAb (HL). This was surprising considering that the S228P mutation has been engineered into multiple therapeutic mAbs to prevent the formation of half-mAb and Fab-arm exchange. However, this result is consistent with the observation that hinge stabilized IgG4 mAbs containing the S228P mutation can undergo Fab-arm exchange, albeit under more reducing conditions than those required for unmodified IgG4s (Labrijn et al., Nat. Biotechnol. 27: 767-71 (2009); Stubenrauch et al., Drug Metab. Dispos. 38(1): 84-91 (2010)). Additionally, these results demonstrate that half-mAb formation and stability towards reduction are not correlated in all instances. The IgG4 mAbs were more stable towards reduction than IgG1s yet the IgG1s formed comparatively low levels of half-mAb. The results in FIG. 2 are depicted in FIG. 3 illustrating the major intermediate species formed for different mAb types as they are reduced by the thioredoxin system.

### Example 2: Hinge Mutations Prevent Half-mAb Formation and Increase Stability Towards Reduction

The heavy chain hinge sequence for an IgG1, IgG2, and IgG4 were compared (Table1). IgG1 and IgG2 mAbs both contain a proline at position 228 while IgG4 mAbs contain a serine. The S228P mutation has been widely used across the industry to prevent Fab-arm exchange and was therefore included in the evaluation.

TABLE 1
Amino acid sequence comparison of the hinge region for IgG1, IgG2, and
IgG4 antibodies using EU numbering. The positions of the mutations
evaluated are indicated in bold text.

| | Position | | | | | | | | | | | | | | | |
|---|---|---|---|---|---|---|---|---|---|---|---|---|---|---|---|---|
| | 215 | 216 | 217 | 218 | 219 | 220 | 221 | 222 | 223 | 224 | 225 | 226 | 227 | 228 | 229 | 230 |
| IgG1 HC | V | E | P | K | S | C | D | K | T | H | T | C | P | P | C | P |
| (SEQ ID | | | | | | | | | | | | | | | | |
| NO: 5) | | | | | | | | | | | | | | | | |
| IgG2 HC | V | E | R | K | C | C | — | — | — | V | E | C | P | P | C | P |
| (SEQ ID | | | | | | | | | | | | | | | | |
| NO: 6) | | | | | | | | | | | | | | | | |
| IgG4 HC | V | E | S | K | Y | G | — | — | — | P | P | C | P | S | C | P |
| (SEQ ID | | | | | | | | | | | | | | | | |
| NO: 7) | | | | | | | | | | | | | | | | |

Two additional novel mutations based on the hinge sequence of an IgG2 mAb were identified. IgG1, IgG2, and IgG4 mAbs contain disulfide bonds at positions 226 and 229, while IgG2s contain additional disulfide bonds at positions 219 and 220. IgG2 mAbs are the most stable towards reduction and it was hypothesized that this is at least in part due to the four disulfide bonds in the hinge region compared to only two hinge disulfide bonds for IgG1 and IgG4 mAbs. Based on the sequence alignment (Table 1), Y219C and G220C mutations were evaluated in an IgG4; each of these mutations add an additional disulfide bond between heavy chains in the hinge region.

TABLE 2
Amino acid sequences of the hinge region for IgG4 antibodies using EU
numbering. The positions of the mutations are indicated in bold text.

| | Position (EU numbering) | | | | | | | | | | | | | | | |
|---|---|---|---|---|---|---|---|---|---|---|---|---|---|---|---|---|
| | 215 | 216 | 217 | 218 | 219 | 220 | 221 | 222 | 223 | 224 | 225 | 226 | 227 | 228 | 229 | 230 |
| IgG4 HC | V | E | S | K | Y | G | — | — | — | P | P | C | P | S | C | P |
| unmodified | | | | | | | | | | | | | | | | |
| SEQ ID | | | | | | | | | | | | | | | | |
| NO: 7) | | | | | | | | | | | | | | | | |
| IgG4 HC | V | E | S | K | C | G | — | — | — | P | P | C | P | S | C | P |
| Y219C | | | | | | | | | | | | | | | | |
| (SEQ ID | | | | | | | | | | | | | | | | |
| NO: 1) | | | | | | | | | | | | | | | | |
| IgG4 HC | V | E | S | K | Y | C | — | — | — | P | P | C | P | S | C | P |
| G220C | | | | | | | | | | | | | | | | |
| (SEQ ID | | | | | | | | | | | | | | | | |
| NO: 2) | | | | | | | | | | | | | | | | |
| IgG4 HC | V | E | S | K | Y | G | — | — | — | P | P | C | P | P | C | P |
| S228P | | | | | | | | | | | | | | | | |
| (SEQ ID | | | | | | | | | | | | | | | | |
| NO: 8) | | | | | | | | | | | | | | | | |
| IgG4 HC | V | E | S | K | C | G | — | — | — | P | P | C | P | P | C | P |
| Y219C + | | | | | | | | | | | | | | | | |
| S228P | | | | | | | | | | | | | | | | |
| (SEQ ID | | | | | | | | | | | | | | | | |
| NO: 9) | | | | | | | | | | | | | | | | |
| IgG4 HC | V | E | S | K | Y | C | — | — | — | P | P | C | P | P | C | P |
| G220C + | | | | | | | | | | | | | | | | |
| S228P | | | | | | | | | | | | | | | | |
| (SEQ ID | | | | | | | | | | | | | | | | |
| NO: 10) | | | | | | | | | | | | | | | | |
| IgG4 HC | V | E | S | K | C | C | — | — | — | P | P | C | P | S | C | P |
| Y219C + | | | | | | | | | | | | | | | | |
| G220C | | | | | | | | | | | | | | | | |
| (SEQ ID | | | | | | | | | | | | | | | | |
| NO: 3) | | | | | | | | | | | | | | | | |
| IgG4 HC | V | E | S | K | C | C | — | — | — | P | P | C | P | P | C | P |
| Y219C + | | | | | | | | | | | | | | | | |
| G220C + | | | | | | | | | | | | | | | | |
| S228P | | | | | | | | | | | | | | | | |
| (SEQ ID | | | | | | | | | | | | | | | | |
| NO: 4) | | | | | | | | | | | | | | | | |

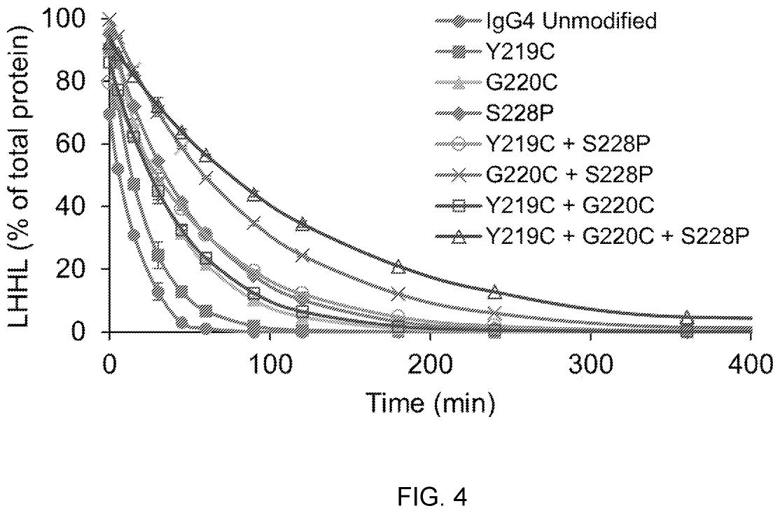

The relative stability of a set of IgG4s (IgG4, IgG4-Y219C, IgG4-G220C, and IgG4-S228P) were examined towards reduction by the thioredoxin system (FIG. 4). The results in FIG. 4 were fit to a first-order exponential decay model to calculate the half-life of each mAb. The half-life of the mAb represents the stability of the mAb towards reduction by the thioredoxin system and comparison of the half-life of the IgG4s with hinge mutations to the unmodified IgG4 provides the relative improvement in stability (Table 3).

TABLE 3
The relative stability of the IgG4s was assessed by
fitting the results in FIG. 4 to a first-order exponential
decay to determine the half-life of each mAb.

| IgG4 Hinge Region | | Fold |
|---|---|---|
| Mutation(s) | 50% Reduced (min) | Improvement |
| Unmodified | 12.3 | NA |
| Y219C | 15.9 | 1.3 |
| G220C | 27.5 | 2.2 |
| S228P | 37.3 | 3.0 |
| Y219C + S228P | 44.0 | 3.6 |
| G220C + S228P | 59.0 | 4.8 |
| Y219C + G220C | 32.1 | 2.6 |
| Y219C + G220C + S228P | 83.9 | 6.8 |
| IgG1λ | 3.2 | 0.3 |
| IgG2λ | 41.7 | 3.4 |

The results show that the Y219C mutation provided only minimal improvement in stability while G220C and S228P mutations respectively provide greater than 2-fold and 3-fold increases in stability towards reduction. Next, the intermediate species formed as these four mAbs were reduced were evaluated to determine if there were differences in the amount of half-mAb formed. In accord with earlier results (FIG. 2), both the unmodified IgG4 and IgG4-S228P formed mainly half-mAb as they were reduced by the thioredoxin system. Interestingly, as compared to the wild type IgG4, the Y219C mutation nearly eliminated the formation of half-mAb while the G220C mutation reduced the amount of half-mAb. In summary, the Y219C mutation provided only a minimal increase in stability towards reduction but nearly eliminated half-mAb formation, the G220C mutation both increased the stability of the mAb towards reduction and reduced half-mAb formation, and the S228P mutation increased the stability of the mAb towards reduction but did not impact the amount of half-mAb formed.

### Example 3: Effects of the Hinge Mutations are Additive

Combinations of the hinge mutations were evaluated to determine if the beneficial effects of each individual mutation are additive when combined in the same molecule.

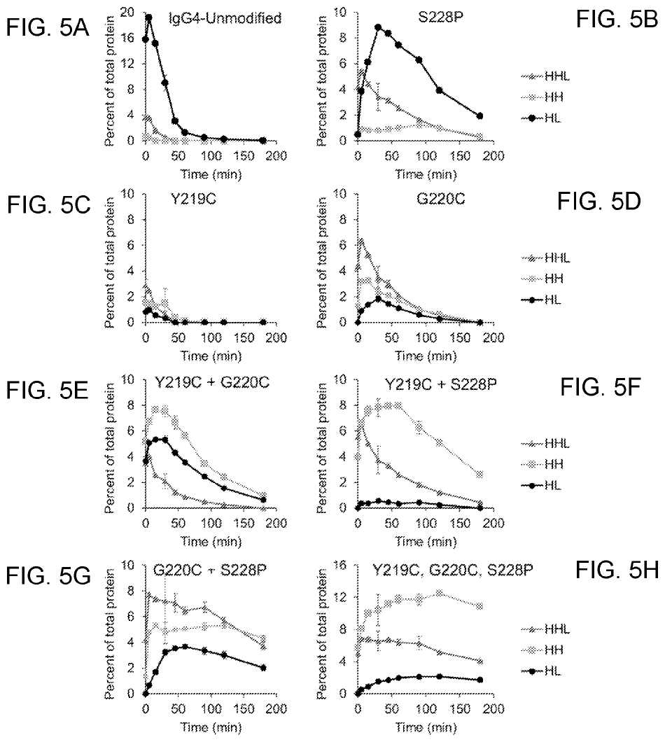

The first double mutant evaluated contained both the Y219C and S228P mutations. Individually, the Y219C mutation prevented half-mAb formation with minimal increase in stability towards reduction and the S228P mutation increased stability towards reduction with no change in the amount of half-mAb formed. The IgG4-Y219C+S228P significantly improved stability towards reduction (FIG. 4, Table 3) and reduced half-mAb formation as compared to the unmodified IgG4 (FIG. 5). Importantly, the IgG4-Y219C+S228P had slightly improved stability towards reduction as compared to the IgG4-S228P mAb and comparable half-mAb formation compared to the IgG4-Y219C. These results show that the effects of the mutations are additive; addition of the Y219C mutation to the IgG4 containing the S228P mutation slightly increased the stability of the mAb towards reduction while also nearly eliminating half-mAb formation.

The second double mutant evaluated contained both the G220C and S228P mutations. Individually, the G220C mutation increased stability towards reduction with reduced half-mAb formation and the S228P mutation increased stability towards reduction with no change in the amount of half-mAb formed. The IgG4-G220C+S228P had significantly improved stability towards reduction (FIG. 4, Table 3) and reduced half-mAb formation compared to the unmodified IgG4 (FIG. 5). Importantly, the IgG4-G220C+S228P had greater stability towards reduction than either the G220C or S228P IgG4s individually. Additionally, the IgG4-G220C+S228P had reduced half-mAb formation, as was observed with the IgG4-G220C. These results provide further evidence that the effects of the mutations are additive; the G220C mutation increases stability towards reduction and reduces half-mAb formation and the S228P mutation increases stability towards reduction.

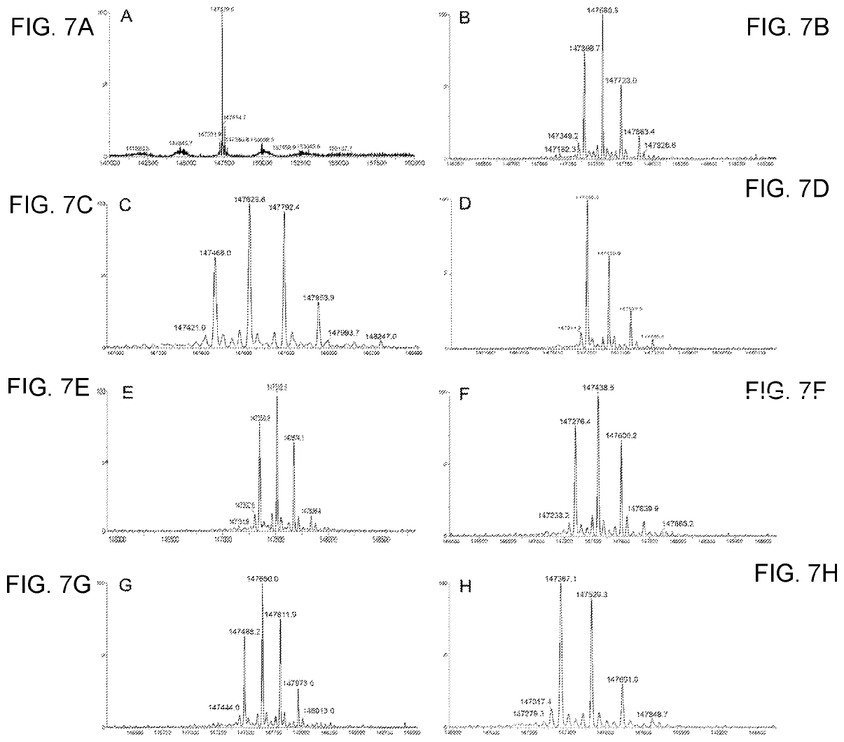

The last double mutant evaluated contained both the Y219C and G220C mutations. Individually, the Y219C mutation prevented half-mAb formation with minimal increase in stability towards reduction and the G220C mutation increased stability towards reduction with reduced half-mAb formation. The IgG4-Y219C+G220C had significantly improved stability towards reduction (FIG. 4, Table 3) and reduced half-mAb formation compared to the unmodified IgG4 (FIG. 5). The IgG4-Y219C+G220C had improved stability towards reduction as compared to the IgG4-G220C, however, the amount of half-mAb formed was also similar to the IgG4-G220C. In this case, the addition of the Y219C mutation to the G220C mAb did provide minor improvement in stability, however the additional decrease in half-mAb formation was not observed. Intact mass spectrometry analysis of this molecule revealed only two disulfide bonds formed in the hinge region rather than the expected four (Table 4, Table 5, FIGS. 7A-7H).

TABLE 4
Summary of the effect of each mutation on the number
of hinge disulfide bonds, relative stability towards
reduction, and half-mAb formation.

| | Additional | Increased | | |
|---|---|---|---|---|
| | Hinge | Stability | Decrease in | Decrease in |
| IgG4 Hinge Region | Disulfide | Toward | half-mAb | Fab-arm |
| Mutation(s) | Bond(s) | Reduction | Formation | Exchange |
| Y219C | 1 | ± | +++ | ++ |
| G220C | 1 | + | ++ | +++ |
| S228P | 0 | + | − | + |
| Y219C + S228P | 1 | + | +++ | +++ |
| G220C + S228P | 1 | ++ | ++ | +++ |
| Y219C + G220C | 0 | ++ | + | ++ |
| Y219C + | 2 | +++ | +++ | +++ |
| G220C + S228P | | | | |

TABLE 5
Summary of the intact mass spectrometry analysis of the IgG4 mAbs with
and without hinge region mutations.

| | | Additional | | | | |
|---|---|---|---|---|---|---|
| IgG4 | | Hinge | Expected | Detected | | |
| Hinge Region | Peak | Disulfide | Mass | Mass | Accuracy | |
| Mutation | ID | Bonds | (Da) | (Da) | (ppm) | Da |
| WT | GOF | 0 | 147378.5 | 147379.6 | −7.5 | −1.1 |
| S228P | GOF | 0 | 147398.6 | 147398.7 | −0.7 | −0.1 |
| G220C | GOF | 1 | 147468.7 | 147468.0 | 4.7 | 0.7 |
| Y219C | GOF | 1 | 147256.4 | 147255.9 | 3.4 | 0.5 |
| Y219C + G220C | GOF | 0 | 147346.6 | 147350.9 | −2.0 | −0.3 |
| Y219C + S228P | GOF | 1 | 147276.5 | 147276.4 | 0.7 | 0.1 |
| G220C + S228P | GOF | 1 | 147488.7 | 147488.2 | 3.4 | 0.5 |
| Y219C + | GOF | 2 | 147366.7 | 147367.1 | −2.7 | −0.4 |
| G220C + S228P | | | | | | |

The last mAb evaluated contained all three mutations. The IgG4-Y219C+G220C+S228P had the greatest overall stability towards reduction (FIG. 4, Table 3) and also formed very little half-mAb (FIG. 5). The effects of the three mutations for this mAb were additive while in the case of the double mutation, IgG4-Y219C+G220C, they were not. Intact mass spectrometry analysis of the triple mutation mAb demonstrated that all four hinge disulfide bonds were formed. All mAbs in this study, except for IgG4-Y219C+G220C, had the intended number of hinge disulfide bonds (Table 4, Table 4, FIG. 7). Previous analysis of IgG4 mAbs with the S228P mutation suggested that the increased stability is the result of a conformation change in the hinge region due to the less flexible amino acid proline amino acid in place of serine. It is likely that in this case the hinge conformational change caused by the S228P mutation was required for the proper alignment and formation of four hinge disulfide bonds in the IgG4-Y219C+G220C+S228P.

### Example 4: Hinge Mutations do not Affect Antigen Binding, FcRn Binding, or Fc Effector Function

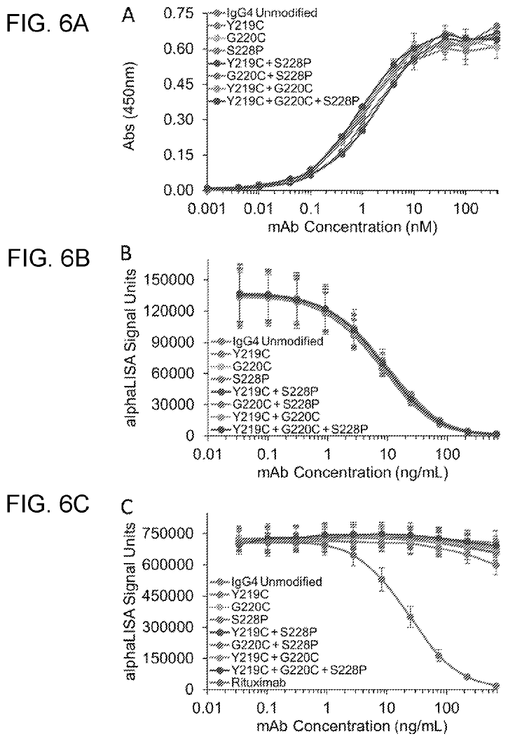

The three mutations evaluated were all in the hinge region and therefore not expected to impact antigen binding. However, it is possible that the additional disulfide bonds in the hinge region of an IgG4 could alter the conformation of the mAb to impair antigen binding. Thus, antigen binding for all seven mAbs with hinge region mutations was compared to the unmodified IgG4 in an ELISA assay. As shown in FIG. 6A, all seven mAbs demonstrated similar antigen binding to the unmodified IgG4. This result demonstrates that these mutations can be incorporated into an IgG4 without affecting antigen binding.

Binding to the neonatal Fc receptor (FcRn) provides IgGs with long serum half-lives, enabling monthly or even less frequent dosing (Pyzik et al., J. Immunol. 194: 4595-603 (2015)). The FcRn binding site is on the heavy chains between the CH3 and CH2 domains and was therefore not predicted to be impacted by the hinge mutations. However, due to the importance of FcRn binding to the efficacy of therapeutic mAbs, it was critical to demonstrate that the hinge mutations do not inhibit FcRn binding. As the results in FIG. 6B demonstrate, all the IgG4s with hinge mutations have similar binding to FcRn as the unmodified IgG4. This result demonstrates that the hinge mutations do not reduce FcRn binding in vitro. Additionally, as the Fab portion remains unchanged, the hinge mutations are unlikely to adversely impact the pharmacokinetics as compared to the unmodified IgG4.

To confirm that the hinge modifications are unlikely to increase ADCC activity, FcγRIIIa binding was evaluated. The results in FIG. 6C demonstrate negligible FcγRIIIa binding for all IgG4s evaluated in this study. Thus, the results shown in FIG. 6 demonstrate that the hinge mutations are unlikely to affect Fc effector function in vivo.

### Example 5: Hinge Mutations Decrease Fab-Arm Exchange

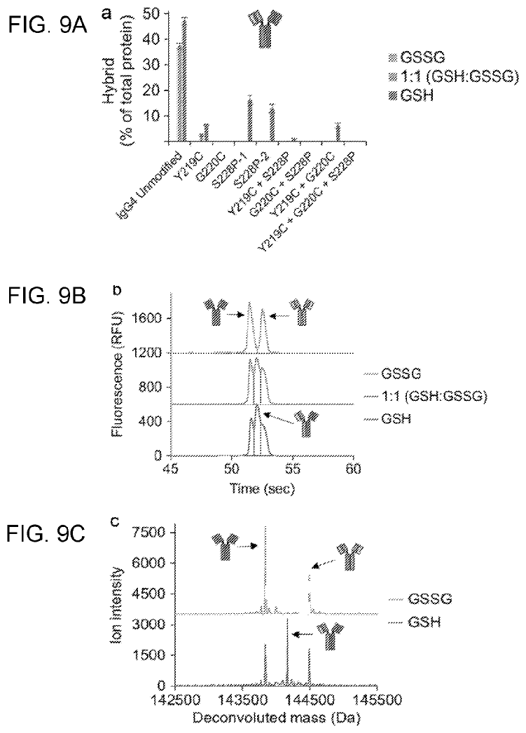

Fab-arm exchange for each IgG4 mAb was evaluated under different redox conditions: (1) oxidizing; (2) mildly reducing; and (3) reducing. Oxidizing conditions were created using oxidized glutathione (GSSG). Mildly reducing conditions were created using a 1:1 ratio of oxidized glutathione and reduced glutathione (GSSG:GSH). Reducing conditions were created using reduced glutathione (GSH). Each IgG4 was separately incubated with an unmodified IgG4 under the different redox conditions. Fab-arm exchange was measured by quantifying the amount of hybrid mAb formation as a percent of total protein using capillary electrophoresis (FIG. 9A) and confirmed using mass spectrometry (FIG. 9C).

The results in FIG. 9A show that all IgG4 mAbs with hinge mutations had significantly reduced Fab-arm exchange compared to the unmodified IgG4. The IgG4-Y219C mAb showed low levels of Fab-arm exchange in the mildly reducing condition that increased in the more reducing GSH condition. The IgG4-G220C mAb showed undetectable levels of Fab-arm exchange under all redox conditions. The IgG4-S228P mAb showed undetectable levels of Fab-arm exchange in oxidizing and mildly reducing conditions, but moderate levels of Fab-arm exchange in the more reducing GSH condition. A second IgG4 with the S228P mutation (S228P-2) was included for comparison and yielded similar results.

Moreover, the combination of hinge mutations showed additive effects in decreasing Fab-arm exchange. The IgG4-Y219C+S228P mAb showed a slight increase in stability toward reduction compared to the IgG4-S228P mAb, and lower Fab-arm exchange than either the IgG4-Y219C or IgG4-S228P mAbs individually. The IgG4-G220C+S228P mAb had undetectable levels of Fab-arm exchange across all redox conditions, in line with the IgG4-G220C mAb. The IgG4-Y219C+G220C mAb had undetectable Fab-arm exchange in oxidizing and mildly reducing conditions, but the level of Fab-arm exchange in the most reducing condition was similar to the levels observed with the IgG4-Y219C mAb under the same conditions. The IgG4-Y219C+G220C+S228P mAb showed the greatest stability toward reduction and undetectable Fab-arm exchange across all redox conditions. These results show the effects of hinge mutations are additive in decreasing the amount of Fab-arm exchange.

1. An IgG4 antibody comprising two heavy chains, wherein each of the heavy chains comprises a human IgG4 constant region comprising at least one mutation at residue 219 or 220 of the heavy chain according to EU index of numbering.

2. The IgG4 antibody of claim 1, wherein the human IgG4 constant region comprises a cysteine at residue 219 of the heavy chain according to EU index of numbering.

3. The IgG4 antibody of claim 2, wherein the human IgG4 constant region comprises a hinge region comprising an amino acid sequence of SEQ ID NO: 1.

4. The IgG4 antibody of claim 1, wherein the human IgG4 constant region comprises a cysteine at residue 220 of the heavy chain according to EU index of numbering.

5. The IgG4 antibody of claim 4, wherein the human IgG4 constant region comprises a hinge region comprising an amino acid sequence of SEQ ID NO: 2.

6. The IgG4 antibody of claim 1, wherein: (i) the human IgG4 constant region comprises a cysteine at residue 219 and a cysteine at residue 220 of the heavy chain according to EU index of numbering: (ii) the human IgG4 constant region comprises a hinge region comprising an amino acid sequence of SEQ ID NO: 3; and/or (iii) the human IgG4 constant region further comprises a proline at residue 228 of the heavy chain according to EU index of numbering.

7. (canceled)

8. (canceled)

9. An IgG4 antibody comprising two heavy chains, wherein each of the heavy chains comprises a human IgG4 constant region comprising a mutation at residues 219, 220, and 228 of the heavy chain according to EU index of numbering.

10. The IgG4 antibody of claim 9, wherein; (i) the antibody comprises a cysteine at residue 219, a cysteine at residue 220, and a proline at residue 228 of the heavy chain according to EU index of numbering; and/or (ii) the human IgG4 constant region comprises a hinge region comprising an amino acid sequence of SEQ ID NO: 4.

11. (canceled)

12. The IgG4 antibody of claim 1, wherein the IgG4 antibody comprises two light chains.

13. The IgG4 antibody of claim 1, wherein the antibody is a human or humanized antibody.

14. The IgG4 antibody of claim 1, wherein the antibody specifically binds erythropoietin, β-amyloid, thrombopoietin, interferon-α (2a and 2b), interferon-β (Ib), interferon-γ, TNFR I (CD120a), TNFR II (CD120b), IL-IR type 1 (CD121a), IL-IR type 2 (CD121b), IL-2, IL2R (CD25), IL-2R-β (CD123), IL-3, IL-4, IL-3R (CD123), IL-4R (CD124), IL-5R (CD125), IL-6R-α (CD126), IL-6R-β (CD130), IL-8, IL-10, IL-I1, IL-15, IL-15BP, IL-15R, IL-20, IL-21, TCR variable chain, RANK, RANK-L, CTLA4, CXCR4R, CCR5R, TGF-β1, -β2, -β3, G-CSF, GM-CSF, MIF-R (CD74), M-CSF-R (CD115), GM-CSFR (CD116), soluble FcRI, sFcRII, sFcRIII, FcRn, Factor VII, Factor VIII, Factor IX, VEGF, α-4 integrin, Cd11a, CD18, CD20, CD38, CD25, CD74, FcαRI, FcεRI, acetyl choline receptor, fas, fasL, TRAIL, hepatitis virus, hepatitis C virus, envelope E2 of hepatitis C virus, tissue factor, a complex of tissue factor and Factor VII, EGFr, HER2, CD4, CD28, VLA-I, 2, 3, or 4, LFA-I, MAC-I, 1-selectin, PSGL-I, ICAM-I, P-selectin, periostin, CD33 (Siglec 3), Siglec 8, TNF, CCL1, CCL2, CCL3, CCL4, CCL5, CCLU, CCL13, CCL17, CCL18, CCL20, CCL22, CCL26, CCL27, CX3CL1, LIGHT, EGF, VEGF, TGFα, HGF, PDGF, NGF, complement, MBL, factor B, a Matrix Metallo Protease, CD32b, CD200, CD200R, OX-40, Killer Immunoglobulin-Like Receptors (KIRs), NKG2D, leukocyte-associated immunoglobulin-like receptors (LAIRs), Iv49, PD-L2, CD26, BST-2, MLL-IAP (melanoma inhibitor of apoptosis protein), cathepsin D, CD40, CD40R, CD86, a B cell receptor, CD79, PD-1, or a T cell receptor.

15.-20. (canceled)

21. The IgG4 antibody of claim 1, wherein the antibody is a bispecific antibody.

22. (canceled)

23. An antibody-drug conjugate comprising the IgG4 antibody of claim 1 and a drug.

24. An antibody-peptide conjugate comprising the IgG4 antibody of claim 1 and a peptide.

25. An antibody-protein conjugate comprising the IgG4 antibody of claim 1 and a protein.

26. A method of treating a subject having a disease or condition comprising administering to the subject a therapeutically effective amount of the IgG4 antibody of claim 1 or a conjugate thereof.

27. The method of claim 26, wherein the disease or condition is selected from the group consisting of: cancer, a neurological disease, a neurodegenerative disease, an autoimmune disease, a cardiovascular disease, a metabolic disease, a respiratory disease, and an infection.

28.-42. (canceled)

43. A method of in vitro diagnosis of a disease or a condition in a subject using the IgG4 antibody of claim 1 or a conjugate thereof.

44. A pharmaceutical composition comprising:
(a) a therapeutically effective amount of the IgG4 antibody of claim 1 or a conjugate thereof; and
(b) a pharmaceutically acceptable carrier.

45. An isolated nucleic acid molecule encoding the antibody of claim 1.

46. A vector comprising the nucleic acid molecule of claim 45.

47. An isolated host cell comprising the vector of claim 46.
